(19) United States
(12) Patent Application Publication
(10) Pub. No.: US 2026/0348755 A1
(43) Pub. Date: Nov. 5, 2026

(54) SYSTEM FOR MONITORING RECEPTACLE FILL LEVEL

(71) Applicant: CareFusion 303, Inc., San Diego, CA (US)

(72) Inventors: Hannah Thompson, San Diego, CA (US); Serena Agrawal, San Diego, CA (US); Kristin Rominger, San Diego, CA (US); Hadley Heitzman, San Diego, CA (US); Nicole Estrich, San Diego, CA (US)

(21) Appl. No.: 19/123,706

(22) PCT Filed: Nov. 1, 2022
(86) PCT No.: PCT/US2022/048583

§ 371 Date: Apr. 23, 2025

(51) Int. Cl. G01F 23/32; G01F 23/00; G01F 23/56; G01F 23/76; G06Q 10/04; G06Q 10/0631; G06Q 10/30; G16H 40/20

(52) U.S. Cl. CPC G01F 23/32; G01F 23/0007; G01F 23/56; G01F 23/76; G06Q 10/04; G06Q 10/06312; G06Q 10/30; G16H 40/20

(57) ABSTRACT

A method may include receiving receptacle data associated with filling levels of one or more receptacles of depositing systems located at a facility. A request to deposit an item is received. A receptacle of the one or more receptacles is identified, based at least on the receptacle data for receiving the item. The receptacle includes a bar having an adjustable feature that adjusts in response to a change in a fill level of the receptacle and one or more sensors configured to detect the adjustable feature of the bar. A user interface is generated to displaying the first receptacle as available to receive the item.

### TECHNICAL FIELD

The subject matter described herein relates generally to systems to monitor receptacle fill levels and more specifically to systems for generating device control messages based on remote detection of fill levels of receptacles in a facility.

### BACKGROUND

Using receptacles as item collection points distributed throughout a facility instead of centrally depositing items can optimize an item return process. The filling level of receptacles can vary depending on multiple factors associated with respective receptacles. For example, items can be deposited in receptacles at varying frequencies and in different amounts, depending on a number of people using particular portions of facilities and depending on events occurring within portions of the facilities. The fluctuations in deposition of items in receptacles can completely fill the receptacles, making them unusable, before a scheduled emptying operation of the full receptacle. If a receptacle is full, item handlers, who intended to deposit items in the full receptacle, would have to look for other available receptacles to deposit the items. The longer a receptacle is full and unusable, the more it would negatively impact the item return process before a preset a scheduled emptying operation of the full receptacle is performed.

### SUMMARY

Systems, sensor assemblies, and articles of manufacture, are provided for using a system for detection of receptacle fill levels in a facility to optimize an emptying schedule of receptacles to minimize (avoid) unusable receptacle time.

In one aspect, a computer-implemented method includes: receiving, by one or more processors, receptacle data associated with filling levels of one or more receptacles of depositing systems located at a facility, receiving, by the one or more processors, a request to deposit an item, identifying, by the one or more processors and based at least on the receptacle data, a first receptacle of the one or more receptacles for receiving the item, and generating, by the one or more processors, a user interface displaying the first receptacle as available to receive the item.

In some variations, one or more features disclosed herein including the following features can optionally be included in any feasible combination. The computer-implemented method can further include detecting, by the one or more processors, depositing of the item at the first receptacle. The computer-implemented method can further include receiving, by the one or more processors, an updated filling level of the first receptacle after detection of disposal of the item at the first receptacle. The computer-implemented method can further include predicting, by the one or more processors, a future filling level of the one or more receptacles of the depositing systems in the facility by applying a machine learning model trained to process the receptacle data associated with filling levels and historical receptacle data, and using the future filling level to update threshold values by applying the machine learning model trained to process the receptacle data associated with filling levels and historical receptacle data. The historical receptacle data can include a frequency of receptacle use, a disposed item type, one or more facility events, and an occupancy level of an associated facility. The computer-implemented method can further include generating, by the one or more processors, a receptacle emptying plan based on the future filling level of the one or more receptacles of the depositing systems in the facility. The computer-implemented method can further include providing, by the one or more processors, for display the receptacle emptying plan. The receptacle emptying plan includes a schedule to empty the one or more receptacles of the depositing systems in the facility within a time period. The receptacle data can include a receptacle bar feature that is variable with a filling level of a respective receptacle, the receptacle bar feature including at least one of a length, a position, and an angle of the receptacle bar. The request to deposit the item can be generated by a second depositing system including a second receptacle and identifying the first receptacle of the one or more receptacles for depositing the item includes: determining, by the one or more processors, a distance between the first receptacle and the second receptacle, the second receptacle having a second filling level above a threshold and the first receptacle having a first filling level below the threshold. The computer-implemented method can further include generating, by the one or more processors, an alert indicating that the second receptacle has the second filling level above the threshold. The computer-implemented method can further include generating, by the one or more processors, an alert indicating one or more nonconformities of filling levels of one or more receptacles. The one or more nonconformities are associated to mismatching receptacle filling data detected by a plurality of sensors or a mismatch between detected receptacle filling data and predicted receptacle filling data. The receptacle data associated with the filling levels of the one or more receptacles is detected by one or more sensors including at least one of a pressure sensor, a camera, a magnetic sensor, a radio frequency sensor, and a light sensor. The item can include an identifier code, and wherein the computer-implemented method further includes receiving, by the one or more processors, the identifier code from a scanning device associated with the first receptacle. The computer-implemented method can further include enabling, by the one or more processors, access to the first receptacle by activating an opening of the first receptacle for depositing the item. The depositing systems can include a user interface, wherein the user interface provides information and/or instructions related to the second receptacle selected for disposing the item. The depositing systems can include a medication depositing system. The instruction to proceed with depositing the item at the first receptacle includes an indication of a location of the first receptacle. Identifying can be based on a physical characteristic of the item relative to an available volume of the one or more receptacles associated with the filling levels of the one or more receptacles. The user interface includes a map of the one or more receptacles and the receptacle data associated with the filling levels of the one or more receptacles.

In another aspect, a depositing system includes a receptacle configured to store one or more items, the receptacle including a bar having an adjustable feature that adjusts in response to a change in a fill level of the receptacle and one or more sensors configured to detect the adjustable feature of the bar, a display panel configured to display instructions for depositing the item, and a computing system configured to perform the computer-implemented method including: receiving receptacle data associated with filling levels of one or more receptacles of depositing systems located at a facility, receiving a request to deposit an item, identifying, based at least on the receptacle data, a first receptacle of the one or more receptacles for receiving the item, and generating a user interface displaying the first receptacle as available to receive the item.

In another aspect, a system includes a depositing system including a receptacle configured to store one or more items, at least one data processor associated with the depositing systems, and at least one memory storing instructions which, when executed by the at least one data processor perform the computer-implemented method including: receiving receptacle data associated with filling levels of one or more receptacles of depositing systems located at a facility, receiving a request to deposit an item, identifying, based at least on the receptacle data, a first receptacle of the one or more receptacles for receiving the item, and generating a user interface displaying the first receptacle as available to receive the item. Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to a system for detection of a receptacle fill level having a sensor assembly, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A depicts a diagram illustrating an example of a system for detection of a receptacle fill level, in accordance with some example implementations;

FIG. 1B depicts a diagram illustrating a portion of the example of the system for detection of a receptacle fill level shown in FIG. 1A, in accordance with some example implementations;

FIGS. 2A and 2B depict example of displays that illustrate filling levels of receptacles within a facility, in accordance with some example implementations;

FIG. 3 depicts an example of a receptacle status report, in accordance with some example implementations;

FIG. 4 depicts a flow diagram for optimizing a receptacle emptying schedule, in accordance with some example implementations;

FIG. 5 depicts a block diagram illustrating an example of a computing system, in accordance with some example implementations;

When practical, similar reference numbers denote similar structures, features, or elements.

### DETAILED DESCRIPTION

Implementations of the present disclosure are generally directed to systems for generating control messages based on remote detection of fill levels of receptacles in a facility. More particularly, implementations of the present disclosure are directed to optimizing a receptacle emptying schedule based on receptacle data collected by sensor assemblies of respective receptacles. The sensor assemblies can transmit the receptacle data to a computing system configured to process the receptacle data and to generate a receptacle emptying schedule based on receptacle data. The computing system can also process depositing requests and generate an instruction to deposit the item at a particular available receptacle within the facility based on the receptacle data. A control message may cause a change in the operational state of one or more devices within the facility responsive to a change in fill level. The operational state change may include adjusting configuration of an automated dispensing cabinet or a receptacle based on the fill level. Examples of such adjustments include locking all or a portion of the device, unlocking all or a portion of the device, activating or communicating a human perceivable indicator, changing power state of the device, raising or lowering the frequency of collecting sensor readings from the device, adjusting item par levels (e.g., minimum or maximum quantities), redirecting return of an item from a full receptacle to a proximately located receptacle with capacity to receive the item, redirecting return of an item from a full receptacle to an empty location within the device (e.g., empty drawer or receptacle within a drawer), or the like.

Automatic detection and real-time tracking of fill levels of multiple receptacles (without human intervention) throughout a facility can help with directing item handlers to deposit items within nearby available receptacles and optimizing an emptying schedule of the receptacles to avoid allowing the receptacle to become completely full and therefore unusable. By controlling the quantity of deposited items between available receptacles and by maintaining the fill level of receptacles within usable ranges, the system prevents delays with item deposition, preventing such delays to interfere with a schedule of item handlers (receptacle users). Another advantage of the disclosed system is that the receptacle data is used to predict receptacle fill levels, enabling proactive adjustment of emptying schedule of the receptacles to avoid allowing the receptacle to become completely full and therefore unusable. Accordingly, the monitoring system described herein may enable an accurate automatic detection of fill levels of receptacles, real-time optimization of deposition of items in receptacles within a facility, dynamic configuration of receptacles or other devices within the facility, and adjustment of an emptying schedule of the receptacles.

Implementations of the present disclosure will be described in view of an example context. The example context includes automatic detection and real-time monitoring of fill levels of receptacles located within medical facilities. Within the example context the receptacles can be used, by receptacle users (item handlers), for depositing (or dispensing of) medical items, including medication that would need to be returned to a pharmacy within a set time interval. The fill level monitoring system can prompt receptacle users to deposit items in selected available receptacles (including available space for depositing additional items) that are within a set distance. In the context of a medication management system, only assigned receptacle users (‘receptacle controllers’) are permitted to access the contents in the return receptacle and re-process medications that have been returned. The fill level monitoring system can determine fill levels of receptacles throughout the medical facility and consider historical receptacle data as well as facility events to optimize receptacle emptying schedules. The fill level monitoring system can use optimized receptacle emptying schedules to prompt receptacle controllers to empty the receptacle at appropriate intervals based on optimized receptacle emptying schedules. Adjustment of the receptacle emptying schedule based on receptacle fill levels can prevent the receptacles within a medical facility from becoming full and therefore unusable for the health care providers, who need to return medications to a particular receptacle. The fill level monitoring system can also help prevent receptacle controllers from having to manually check receptacle fullness throughout a medical facility or emptying the receptacles too frequently.

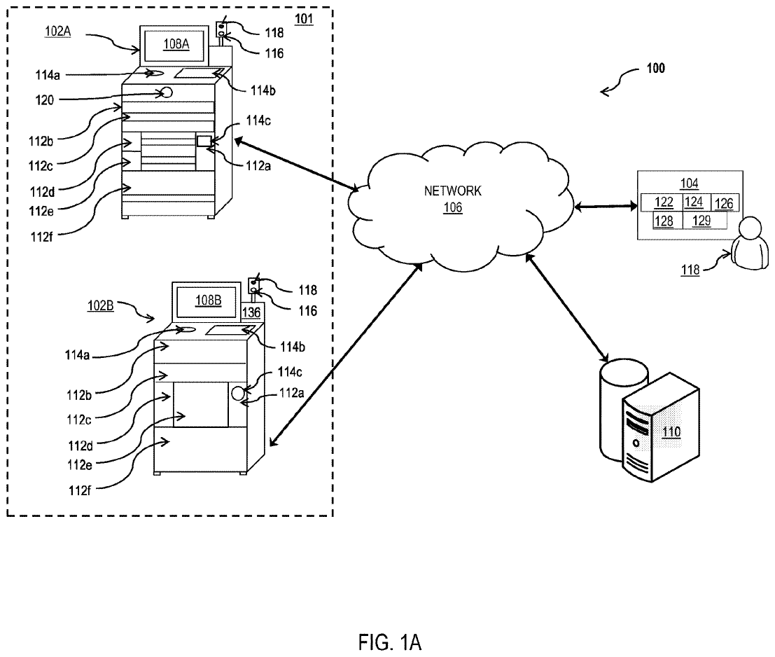

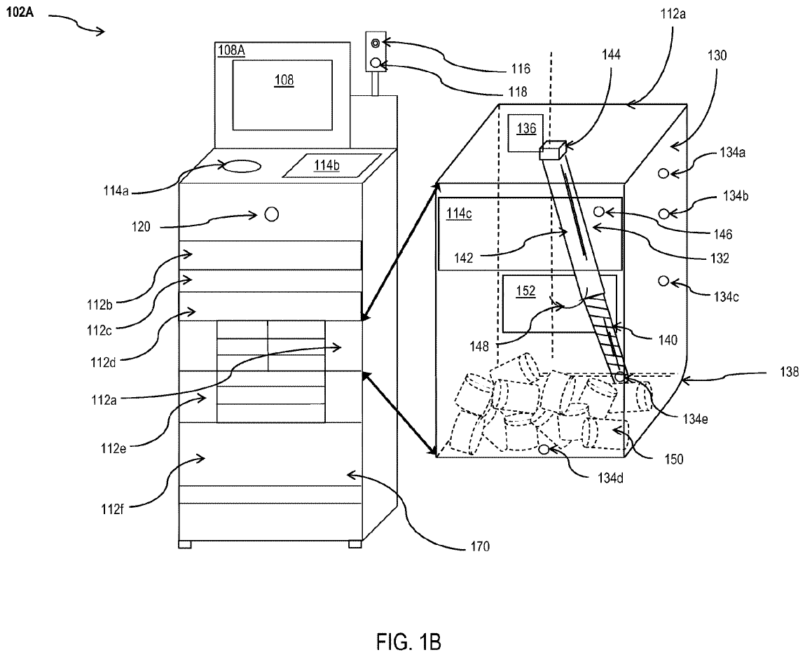

Implementations of the present disclosure are described in further detail herein within an example context of example forms of medical systems. More specifically, and with reference to FIG. 1A, a simplified example of a system for detection and real-time tracking of fill levels of receptacles within a facility 101 is provided. Even though the example facility 101 is illustrated as including two depositing systems 102A, 102B, the facility 101 can include any number (greater than two) of depositing systems. In view of this context, the present disclosure provides integrated visibility of detected fill levels of receptacles to enable real-time adjustment of receptacle emptying schedule. It is appreciated, however, that implementations of the present disclosure are readily applicable in other contexts with other forms of automatic detection and real-time tracking of fill levels of receptacles.

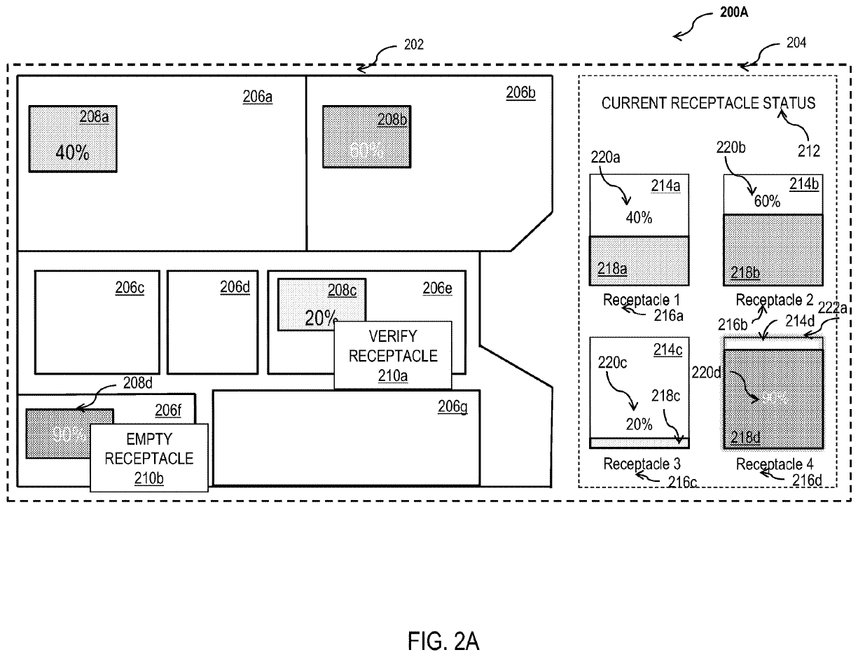

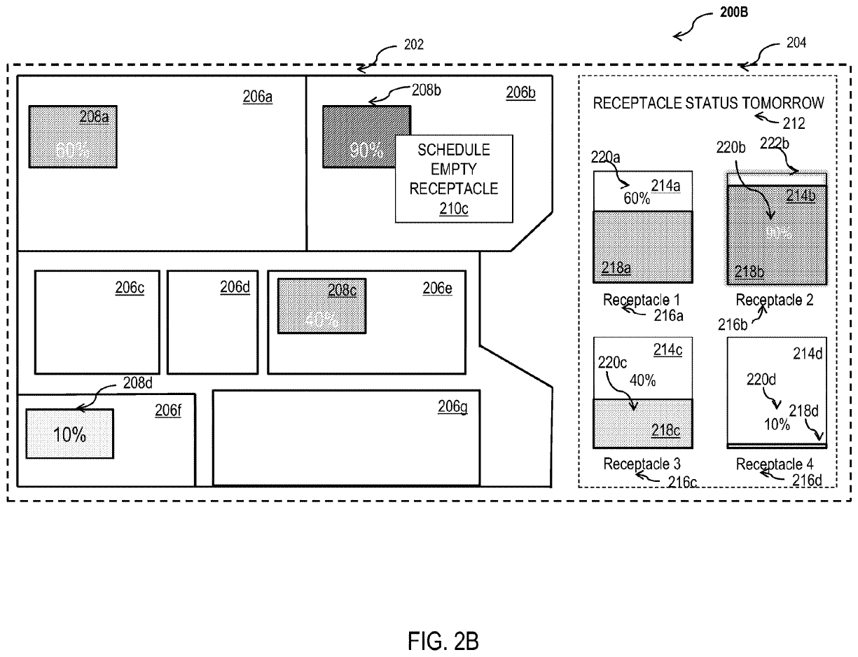

FIG. 1A depicts a diagram illustrating an example system architecture 100 for detection of a receptacle fill level, in accordance with some example implementations. The example system architecture 100 includes a first depositing system 102A, a second depositing system 102B, a user device 104, a network 106, and a data processing system 110. As discussed in further detail herein, each depositing system 102A, 102B includes a user device 108A, 108B configured to determine, display, and transmit receptacle fill level data associated with the receptacles 112a-112f of each of the first and second depositing systems 102A, 102B. For example, the user devices 108A, 108B can transmit receptacle fill level data, over the network 106 to the data processing system 110 for processing and to the user device 104 or any other user device and for presentation or display, as described with reference to FIGS. 2A and 2B. In some implementations, each of the user devices 108A, 108B and to the user device 104 can provide instructions for a receptacle user to deposit items 150 at an available receptacle 112a-112f within the facility 101. Although a single user device 104 is illustrated, it is contemplated that one or more user devices 104 can communicate with each of the first and second depositing systems 102A, 102B through the network 106. Further, data can be transferred between the data processing system 110, each of the first and second depositing systems 102A, 102B, and to the user device 104 through the network 106.

Each depositing system 102A, 102B can be provided as an item depositing system that enables tracking of receptacle fill levels, and the like. Some instances of the depositing system 102A, 102B may be configured as automated medication dispensing cabinets including features similar to, for example, a BD Pyxis MedStation™M from Becton, Dickinson and Company. The depositing systems 102A, 102B can include one or more receptacles 112a-112f. The receptacles 112a-112f can include drawers and/or bins. The receptacles 112a-112f are being monitored to track the fill level of each individual receptacle 112a-112f, using a sensor assembly as described with reference to FIG. 1B. In the example system architecture 100 of FIG. 1A, the first depositing system 102A, and the second depositing system 102B include one or more receptacles 112a that could each be configured to securely receive items 150, when the fill level is below a set threshold (receptacle is not full) and to store the deposited items 150 until the receptacle 112a-112f is emptied by assigned users (receptacle controllers). It is contemplated that each depositing system 102A, 102B can include one or more receptacles 112a-112f, of different shapes and sizes, without being limited to the example arrangement illustrated in FIGS. 1A and 1B. Each of the receptacles 112a-112f can include a sensor assembly configured to monitor a fill level of the respective receptacle 112a-112f, as described with detail to FIGS. 1B, 2A-2C, and 3.

Each depositing system 102A, 102B includes a user (computing) device 108A, 108B that can guide a user through the depositing process, including, for example, authenticating the user, labeling and/or securing the deposited item, and/or depositing the item in a corresponding available receptacle through a deposit point (e.g., a first item type general receiver 114a or a second item type general receiver 114b or an entry mechanisms 114c of a respective receptacle 112a) of the depositing system 102A, 102B. In some implementations, a user can deposit items in a bin without being guided through one or more of the depositing process.

Also shown in the representation of the depositing system 102A, 102B of FIGS. 1A and 1B are a biometrics scanner 116, a sensor 118, and a smart lock 120. The sensor 118 can include a camera, a radio frequency sensor, a scale, and any other type of sensor that can provide volume and weight data associated with the deposited items 150. The data collected by the sensor 118 can be transmitted to the data processing system 110 to be processed, in addition to the receptacle sensor data, to verify an accuracy of receptacle filling level.

The user device 108A, 108B integrated in the depositing system 102A, 102B includes a user interface 108 that may display prompts on the display and/or accept inputs from the user to guide the user through the depositing process, thereby confirming each step is complete, secure, and auditable. The user device 108A, 108B integrated in the depositing system 102A, 102B may provide visual feedback based on images captured by the sensor 118. The visual feedback may allow the user to verify that the depositing system 102A, 102B has a clear image of the item being deposited in selected receptacles 112a-112f that are detected as not being full.

The receptacles 112a-112f may include one or more drawers. The receptacles 112a-112f may include audit receptacles, for example, receptacles of highly controlled substances. The receptacles 112a-112f may each include passages to an interior of the receptacle in a way that optimizes detection of fill level of receptacle (e.g., from a direction that would generate a trigger of a sensor detecting a change in fill level). The receptacles 112a-112f can be configured for depositing items 150 in bulk. The arrangement, as shown in FIGS. 1A and 1B, allows for the collection of different types of items 150 in receptacles 112a-112f for auditing the deposited items 150. Moreover, the drawer arrangement as shown in FIGS. 1A and 1B allows for the items not selected for audit to be collected in the bulk receptacles. In some implementations, the depositing systems 102A, 102B are located at the same facility 101 or at different facilities. In the case of multiple facilities, the facilities can be remotely located from one another, and/or can be located at a common location, or site (e.g., separate departments in a common (the same) building).

In some implementations, each facility 101 includes an associated data processing system 110 that can be located at a site of the facility 101 or it can be remotely located (at a site that is separated from the facility site). Each data processing system 110 can be provided as a server (e.g., a front-end server, a back-end server, a cloud server), and supports the acquisition, storage, modification, and distribution of receptacle fill level information, such as receptacle fill levels, throughout the facility 101 including the depositing systems 102A, 102B. Although the example system architecture 100 includes a data processing system 110 located remotely from the depositing systems 102A, 102B, it is contemplated that the data processing system 110 can be integrated within any or all of the depositing systems 102A, 102B.

In some implementations, the user device 104, 108A, 108B can include a personal computer (PC) (e.g., desktop, laptop, or tablet). Communication between each user devices 104, 108A, 108B and the data processing system 110 can be achieved via a direct connection, or remotely through the network 106 that can include, but is not limited to, a local area network (LAN), a wide area network (WAN), and/or the Internet.

The user device 104 can include any number of example devices. Such example devices include, but are not limited to, a mobile phone, a smartphone, a tablet computing device, a personal digital assistant (PDA), a laptop personal computer (PC), a desktop PC, and/or appropriate combinations thereof. In the depicted example, the user device 104 includes a display 122, a processor 124, memory 126, an input interface 128, and a communication interface 129. The processor 124 can process instructions for execution of implementations of the present disclosure. The instructions can include, but are not limited to, instructions stored in the memory 126 to display graphical information on the display 122. Example displays include, but are not limited to, a thin-film-transistor (TFT) liquid crystal display (LCD), or an organic light emitting diode (OLED) display. The memory 126 stores information within the user device 104. In some implementations, the memory 126 can include a volatile memory unit or units, and/or a non-volatile memory unit or units. In other implementations, removable memory can be provided, and can include, but is not limited to, a memory card. Example memory cards can include, but are not limited to, a secure digital (SD) memory card, a mini-Secure Digital (SD) memory card, a Universal Serial Bus (USB) stick, and the like.

In some implementations, the input user interface 108, 128 can include a keyboard, a touchscreen, a mouse, a trackball, a microphone, a touchpad, and/or appropriate combinations thereof. In some implementations, an audio codec (not shown) can be provided, which receives audible input from a user or other source through a microphone, and converts the audible input to usable digital information. The audio codec can generate audible sound, such as through a speaker that is provided with the user device 104. Example sounds can include sound from voice telephone calls, recorded sound (e.g., voice messages, music files, etc.), and/or sound generated by applications operating on the user device 104.

The user devices 104, 108A, 108B can communicate over the network 106 through a connectivity interface(s). In some implementations, the connectivity interface(s) can include a satellite receiver, cellular network, a Bluetooth system, a Wi-Fi system (e.g., 802.x), a cable modem, a DSL/dial-up interface, a private branch exchange (PBX) system, and/or appropriate combinations thereof. Each of these connectivity interfaces enables data to be transmitted to/from the network 106. In some implementations, the network 106 can be provided as a local area network (LAN), a wide area network (WAN), a wireless LAN (WLAN), a metropolitan area network (MAN), a personal area network (PAN), the Internet, and/or combinations thereof.

In some implementations, each depositing system 102A, 102B includes a sensor assembly 130 to monitor a receptacle fill level of each receptacles 112a-112f, and generates data signals based thereon. As discussed in further detail herein with reference to FIG. 1B, implementations of the present disclosure provide a sensor assembly 130 that includes a bar 132 (including an adjustable feature), one or more sensors 134a-e, and a controller 136.

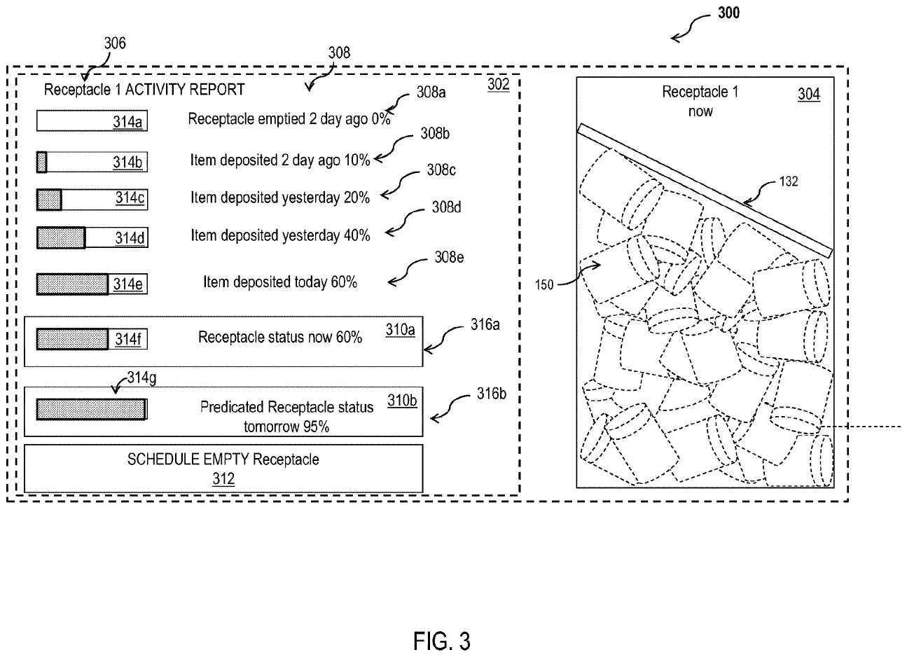

The bar 132 has an adjustable feature (such as a position, an angle, and/or a length) that adjusts in response to a change in a fill level of the receptacle 112a. The bar 132 can be made of an inelastic light weight material (e.g., fiberglass). The bar 132 can be secured to an interior wall of the receptacle 112a by a pivoting joint 144 that enables the bar to rotate (without bending) in response to the change in the fill level of the receptacle 112a (as shown in FIGS. 2 and 3). The bar 132 can include a variable length section 140 and, optionally, a fixed length section 142. The variable length section 140 can become shorter as the receptacle fill level increases and can be configured to automatically extend to maximum length after the receptacle is emptied. The variable length section 140 of the bar 132 can include a telescoping bar, a spring, and/or a telescopic spring. Even though the bar 132 is illustrated as being attached to a central portion of the top horizontal wall of the receptacle 112a, the bar 132 can attached to any top portion of the receptacle 112a, including a top portion of a vertical wall above the entry mechanism 114c. The bar 132 can be attached to the receptacle 112a to enable the bar 132 to form an angle 148 with a vertical axis (e.g., a vertical wall of the receptacle 112a) that increases with an increasing amount of items 150 deposited in the receptacle 112a.

In some implementations, at least a portion of the inner walls of the receptacle 112a can be configured to enable a movement of the bar 132 within the receptacle 112a. For example, a portion of the inner walls of the receptacle 112a can include a fileted edge 138 configured to guide a distal edge of the bar 132 through a transition between two orthogonal surfaces of the receptacle 112a. The fileted edge 138 can include a curved surface with a radius that matches the variable length of the bar 132 as it transitions between two orthogonal (horizontal and vertical) surfaces of the receptacle 112a. The fileted edge 138 can be oppositely located from the entry mechanism 114c, such that the bar 132, when pushed by the deposited items 150 towards the fileted edge 138 and away from the entry mechanism 114c, can rotate and adjust the length to indicate the fill level of the receptacle 112a.

The sensors 134a-e can include a sensory array 134a-134c disposed along a first inner surface of the receptacle 112f, a separate sensor 134d disposed on a second inner surface of the receptacle 112f, and one or more sensors 134e attached to the bar 132 (e.g., an ending portion of the bar 132). The sensory array 134a-134c can include multiple sensors in the array. The number of sensors in the sensory array 134a-134c can vary to give a selected granularity of receptacle fill level detection. The sensor 134c can be separate from the sensory array 134a-134c to generate a second sensor data type that can be used as to verify a filling level of the receptacle as determined based on the data received from the sensory array 134a-134c. The sensors 134a-e can include a gyroscope, a pressure sensor, a camera, a magnetic sensor, a radio frequency sensor, a light sensor, a force sensor, a pressure sensor, or the like. For example, the sensors 134a-c, 134e can generate location data including a location of one or more points of the bar 132 (e.g., a distal end of the bar 132) or the location of the entire bar associated with a position, an angle, and/or a length of the bar 132 and, optionally, based on the angle 148 of the bar 132. The location data generated by the sensors 134a-c, 134e can be used to generate a filling level of a respective receptacle 112a. The sensor 134d can generate weight (force or pressure) data that in combination image data generated by the sensor 118 can also indicate a filling level of a respective receptacle 112a. In some implementations, if the filling level derived based on signals from the sensors 134d and sensor 118 does not match the filling level derived based on signals from the sensors 134a-c, the indicator can be automatically switched to an alert state (display a red color) and an alert can be associated to the displayed receptacle to be verified, as described with reference to FIG. 2A.

The sensors 134a-e can detect signals associated with a filling level of a respective receptacle 112a continuously and/or at various time intervals (e.g., every 10 seconds, 30 seconds, 1 minute, 30 minutes, 1 hour, 12 hours, 24 hours, and the like). In some implementations, the controller 136 controls the time at which the sensors 134a-e measure the bar length, position, and angle 148. The sensors 134a-e may transmit signals of the detected signals (e.g., bar length, position, and bar angle 148) to the controller 136 immediately after the signal detection. In some implementations, the sensors 134a-e can be activated by an activation of an entry mechanism 114c to detect a change in bar length based on a change in receptacle fill level (after deposition of items 150 within the receptacle 112a through the entry mechanism 114c). The entry mechanism 114c can include a slot, a slanted ramp, and a door. The entry mechanism 114c can be generally blocked to prevent access to the corresponding receptacle 112a and can be opened in response to a user's request to deposit items 150 and in response to the sensor assembly indicating that a fill level of the receptacle 112a is below a critical fill level (e.g., receptacle 112a is not full). For example, the controller 136 can be configured to respond to the fill level of the receptacle 112a satisfying one or more thresholds by at least blocking the entry mechanism 114c to prevent additional items from being deposited into the receptacle 112a.

The sensors 134a-e can be configured to transmit the signals indicative of a filling level of a respective receptacle 112a to the controller 136. The controller 136 can transmit the data recorded by the sensors 134a-e to the data processing system 110. In some implementations, the controller 136 can process signals received from the sensors 134a-e to determine the fill level of the receptacle 112a. The controller 136 can be configured to transmit the fill level of the receptacle to the data processing system 110 to optimize the receptacle emptying plan for the entire facility 101. The data processing system 110 can transmit the emptying plan to the user interface 108 and/or an indicator 146 to display the fill level of the receptacle 112a. For example, the controller can be coupled to with the indicator 146 (e.g., LED lights, software) to relay the fullness level of the receptacle 112a to potential receptacle users and receptacle controllers. In some implementations, the indicator 146 can use a color code to indicate the fullness level of the receptacle 112a (e.g., green color indicating that the receptacle includes sufficient empty volume for depositing additional items, orange color indicating that the receptacle includes limited empty volume for depositing additional items, and red color indicating that the receptacle is full and includes insufficient empty volume for depositing additional items).

The controller 136 can be physically coupled to the receptacle 112a or it can be positioned at a location separate from the receptacle 112a. In some implementations, a distal edge of the bar 132 is configured to interact (e.g., through contact) with a first sensor (e.g., sensor 134d) of the one or more sensors 134a-e when the bar 132 is at a first length corresponding to the receptacle being at a first fill level. The controller 136 can determine that the bar 132 is at the first length based on a first signal generated by the first sensor 134d in response to being contacted by the bar 132 at the first length. The distal edge of the bar 132 can be further configured to interact (e.g., through contact) with a second sensor (e.g., sensor 134a, 134b, or 134c) of the one or more sensors when the bar is at a second length corresponding to the receptacle being at a second fill level (e.g., the receptacle 112a being more filled with items 150 than at the first fill level). The controller 136 can determine that the bar 132 is at the second length based on a second signal generated by the first sensor and the second sensor in response to being contacted by the bar at the second length. The controller 136 can determine that the bar 132 is at a third length, greater than the first length and shorter than the second length based on a third signal generated by the first sensor 134d and the second sensor (e.g., sensor 134a, 134b, or 134c).

The controller 136 can be physically coupled to the receptacle 112a or it can be positioned at a location separate from the receptacle 112a (within the respective depositing system 102A). In some implementations, the controller 136 can activate an item settling feature 152 in response to determining that the entry mechanism 114c was activated. The item settling feature 152 can be configured to generate a movement impulse (e.g., vibration, rotation, vertical oscillation) to trigger settling of items 150 deposited within the receptacle 112. The item settling feature 152 can be attached to any vertical and/or horizontal wall of the receptacle 112a. In some implementations, the controller 136 can activate the sensors 134a-e to detect the length of the bar 132 and, optionally, based on the angle 304 of the bar 132, after the items 150 within the receptacle 112 are settled. The controller 136 may translate or convert the sensor measurements to determine the length and angle of the bar and corresponding filling levels of the receptacle 112a. In some implementations, the controller 136 can detect that filling level of the receptacle 112a exceeds a critical level. For example, the controller 136 may compare a detected filling level of the receptacle 112a to a threshold level (e.g., greater than a set percentage of a total height of the receptacle 112). The controller 136 can transmit a signal to the indicator 146 to display an indication of the detected filling level of the receptacle 112a relative to one or more threshold filling levels. In some implementations, Include an facility administrator can set or adjust (automatically set) one or more threshold filling levels, according to intended alerts/limitations associated with the one or more threshold filling levels.

The example sensor assembly 130 can be coupled to an indicator 146 that can indicate the fullness level of the receptacle 112a. The indicator 146 can be attached to an exterior surface of the receptacle 112a to enable visualization of the fill level indication. For example, the indicator 146 can be attached to the exterior surface of the receptacle 112a near to the entry mechanism 114c that is used for depositing items 150.

In some implementations, the indicator 146 can use a color code to indicate the fullness level of the receptacle 112a (e.g., green, orange, and red) or a numerical display indicating the available or used volume of the receptacle as a percentage or a fillable bar icon that matches the available or used volume of the receptacle. For the examples, if one or more of the sensors 134a-d detect that the bar 132 has a particular length that is greater than a threshold length and/or the bar 132 is at a particular angle 148 that is smaller than an angle threshold, which the controller 136 can use to indicate that the receptacle 112 includes sufficient empty volume for depositing additional items. The indicator 146 can use green color indicating that the receptacle includes sufficient empty volume for depositing additional items. In some implementations, if one or more of the sensors 134a-d detect that the bar 132 has a particular length that is equal or smaller than a threshold length and/or the bar 132 is at a particular angle 148 that is equal or greater than an angle threshold, an alert can be generated by the controller 136 to indicate that the receptacle 112 reached a critical fill level and the receptacle 112 should be planned to be emptied within a particular period of time. The indicator 146 can use orange color indicating that the receptacle includes limited empty volume for depositing additional items. In some implementations, if one or more of the sensors 134a-d detect that the bar 132 has a particular length that is equal or smaller than a second threshold length and/or the bar 132 is at a particular angle 148 that is equal or greater than a second angle threshold, an alert can be generated by the controller 136 to indicate that the receptacle 112 reached a maximum fill level (being full and unusable) and the receptacle 112 should be prioritized to be emptied within a particular period of time. The indicator 146 can use red color indicating that the receptacle is full and includes insufficient empty volume for depositing additional items.

FIGS. 2A and 2B illustrate examples of receptacle displays 200A, 200B for an example facility at different times. The receptacle displays 200A, 200B include a facility map 202 and a receptacle status section 204. The facility map 202 can include a schematic map of the facility (e.g., facility 101 described with reference to FIG. 1A).

The facility map 202 can illustrate multiple departments 206a-g of the facility. Each department 206a-g of the facility can correspond to a division type of the facility. Within the context example, the facility departments 206a-g can include an emergency section, a surgery section, a pediatric section, an adult section, an imaging section, a lab section, a utility section, or any other division type corresponding to the mapped facility. At least a portion of the facility departments 206a-g can include receptacles represented in the facility map 202 by receptacle icons 208a-d. The receptacle icons 208a-d can include respective receptacle fill level indicators. As illustrated in FIGS. 2A and 2B, the receptacle fill level indicators of the receptacle icons 208a-d can include a numerical indicator (e.g., a percentage of filled capacity of the receptacle) and/or a color code (e.g., green to red variation, with green representing empty receptacles and red representing filled receptacles or light to dark color variations, with light representing empty receptacles and dark representing filled receptacles).

In some implementations, the receptacle icons 208a-d include a task flag 210a, 210b that can be overlapping or near to a portion of the receptacle fill level indicators. The task flag 210a, 210b can display an action to be performed in association with the corresponding receptacle. The action indicated by the task flag 210a, 210b, 210c can include an action to be taken for maintaining an availability of the associated receptacle, such emptying actions of a receptacle where sensors indicated fill levels exceeding a threshold (as illustrated in FIGS. 2A and 2B) or a verification of a receptacle where multiple sensors indicated nonconformities (non-matching) fill levels (as illustrated in FIG. 2A for the receptacle icons 208d). The emptying actions can include instructions to immediately empty a particular receptacle that can be unusable because of being full (as illustrated in FIG. 2A) or scheduling an emptying operation of a particular receptacle with a fill level exceeding a particular threshold (e.g., 80% of capacity) (as illustrated in FIG. 2B for the receptacle icons 208b).

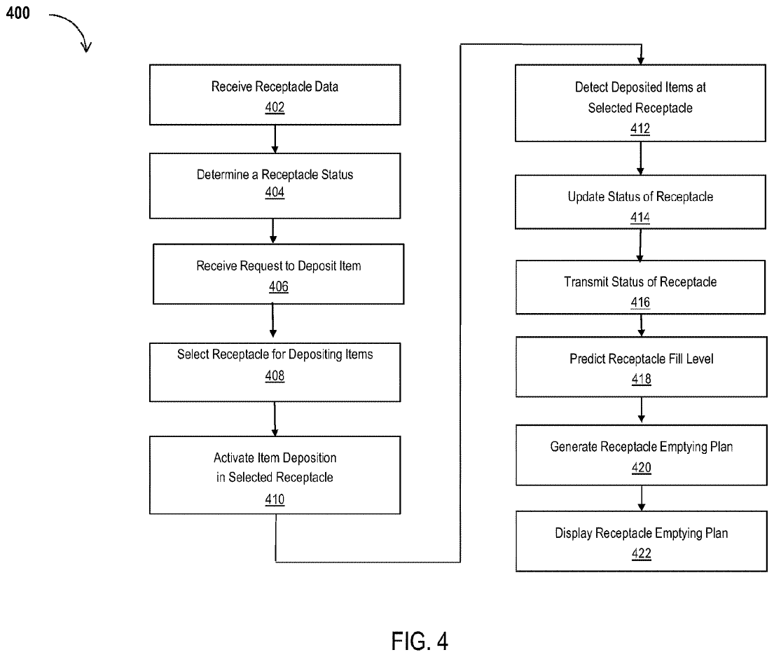

The receptacle status section 204 can include a textual identifier 212 indicating a time corresponding to estimated receptacle fill levels that are being displayed. The time icon can be activated by a user to select a different time than the one currently displayed. The time can include a present time (as illustrated in FIG. 2A), a past time or a predicted future time (as illustrated in FIG. 2B). The receptacle fill levels for current and past times can be derived based on sensor data, as described with reference to FIGS. 1 and 4. The receptacle fill levels for future times can be derived using a machine learning model, current sensor data indicating current fill levels, receptacle historical data, and associated events (e.g., facility events) as described with reference to FIGS. 1 and 4.

The receptacle status section 204 can include fill level icons 214a-d for each receptacle within a (portion of) the facility. Each fill level icons 214a-d has an associated receptacle identifier 216a-d. The receptacle identifier 216a-d can include a numerical identifier and/or textual (name) identifier that can be associated with a department within which the receptacle in positioned. The fill level icons 214a-d can include a color code 218a-d (e.g., green to red variation, with green representing empty receptacles and red representing filled receptacles or light to dark color variations, with light representing empty receptacles and dark representing filled receptacles) and/or a numerical indicator 220a-d (e.g., a percentage of filled capacity of the receptacle). In some implementations, the fill level icons 214a-d of receptacles that have a fill level exceeding a set threshold can be displayed using an added highlighting feature 222a, 222b indicating an action associated to the receptacle. For example, in FIG. 2A, the fill level icons 214d is displayed with a highlighting feature 222a and in FIG. 2B, the fill level icons 214b is displayed with a highlighting feature 222b indicating an action associated to the receptacle. Any of the receptacle icons 208a-d and the fill level icons 214a-d can be selected by a user to visualize a status report associated with the selected receptacle, as illustrated in FIG. 3.

FIG. 3 depicts an example of a receptacle status report 300. The receptacle status report 300 can include an activity report 302 and a visualization panel 304. The activity report can include a label including a receptacle identifier 306 (e.g., matching the receptacle identifier of the receptacle selected to be reviewed from the receptacle displays (e.g., receptacle displays 200A, 200B described with reference to FIGS. 2A and 2B). The activity report can include an itemized list 308 of activities 308a-e, a current receptacle status 310a, a predicted receptacle status 310b and an action control icon 312. The itemized list 308 of activities 308a-e can be set to start the list 308 with a first activity 308a associated with a selected event (e.g., last time the selected receptacle was emptied) or a particular time (that can be selected by the user or preset in the system). The itemized list 308 of activities 308a-e can be set to include a most recent activity 308e associated with a selected event (e.g., items deposited in the receptacle). In some implementations the current receptacle status 310a can be displayed within or outside the itemized list 308. The itemized list 308 of activities 308a-e can include receptacle fill level icons 314a-e in parallel with respective textual labels, each label including a numerical indicator of the respective receptacle fill level.

The current receptacle status 310a can be displayed with a highlighting feature 316a that differentiates the current receptacle status 310a from the activities 308a-e. The current receptacle status 310a can include a receptacle fill level icon 314f in parallel with respective textual label including a numerical indicator of the fill level. The predicted receptacle status 310b can be displayed with a highlighting feature 316b that differentiates the predicted receptacle status 310b from the activities 308a-e. The predicted receptacle status 310b can correspond to a preset time or a selected future time, which can be modified by selecting the time indicator. The action control icon 312 can include a textual label describing a displayed action that can be selected. The displayed action can be associated with the current receptacle fill level status and/or a predicted receptacle fill level status. In some implementations, the action control icon 312 can enable a user to select an action from a list of possible actions (e.g., selection to empty the selected receptacle empty the selected receptacle at a particular time or to prioritize emptying the selected receptacle).

The visualization panel 304 can include a static or animated (video) visual representation of the selected receptacle. The visualization panel 304 can include a representation of the bar 132 (described with reference to FIGS. 1A and 1B) and the deposited items 150. The static representation of the selected receptacle can include an actual image recorded by a camera (sensor) included in the receptacle or a reconstructed image that was generated to visually represent the fill level detected based on the sensor data at a particular time (that can be selected or modified by a user selection). The animated representation of the selected receptacle can include an actual video recorded by a camera (sensor) included in the receptacle or a reconstructed animation that was generated to visually represent the variation in fill level detected based on the sensor data within a set period of time that can correspond to the period of time illustrated in the activity report 302.

FIG. 4 depicts a flowchart illustrating a process 400 for determining a fill level of a receptacle, consistent with implementations of the current subject matter.

At 402, a computing system (e.g., a depositing system 102A, 102B described with reference to FIGS. 1A and 1B or a data processing system 110 described with reference to FIGS. 1A and 1B), receives receptacle data associated with filling levels of one or more receptacles a plurality of receptacles distributed within a facility. The receptacle data includes data detected by one or more sensors (e.g., sensors 134a-e described with reference to FIGS. 1A and 1B) attached to a receptacle. The sensors can include a pressure sensor, a camera, a magnetic sensor, a radio frequency sensor, and a light sensor. The sensors can be configured to detect and transmit one or more types of receptacle data. The receptacle data can include a receptacle bar feature that is variable with a filling level of a respective receptacle. The receptacle bar feature can include a bar length and/or an angle between the bar and a vertical axis intersecting a pivoting element used to attach the bar to a portion of the receptacle. The receptacle data can include additional data (e.g., images, item identifiers associated with item volume and weight, and/or item weight of the items deposited within a respective receptacle). The additional data can be detected independent of the receptacle bar feature data. The additional data can include user authentication (e.g., a user name and password retrieved from a scan of a user identification card using the camera or the sensor, scanning a biometric feature of the user using the camera or the sensor or any combination thereof) that can be associated with an item deposition. In some implementations, the receptacle data is retrieved in response to receiving a user input including a request to deposit one or more items in a receptacle, after items were deposited in a receptacle or at a set frequency, based on a present data generation schedule (e.g., at a set number of times per day).

At 404, a current receptacle status is determined, by the computing system (e.g., a depositing system 102A, 102B described with reference to FIGS. 1A and 1B or a data processing system 110 described with reference to FIGS. 1A and 1B or a controller, such as the controller 136, described with reference to FIGS. 1A and 1B). In some implementations, determining the current receptacle status includes determining a filling level of one or more (e.g., all) receptacles within the facility. The filling level of one or more (e.g., all) receptacles within the facility can be determined by processing the receptacle data (bar feature data and the additional receptacle data). For example, the receptacle filling level can be determined, based on the length of the variable length bar and, optionally, based on the angle of the bar, as detected by the sensors. For example, the measured length of the variable length bar and angle of the bar can each have a set mathematical relationship (that can be defined in a lookup table based on a receptacle geometry type) with the occupied (or remaining available) volume associated with deposited items. Each of the measured length of the variable length bar and angle of the bar can be separately (or together) used to estimate the fill level. For example, an average of the two fill level estimates can be used (if a difference between the two estimates is not greater than a set threshold) to determine the current fill level (e.g., provided as a percentage of total available volume) with an increased accuracy of the fill level estimate. The estimated receptacle fill level derived from the receptacle bar feature can be compared to an estimated receptacle fill level derived based on the additional data (e.g., item weight and item volume derived from item identifiers and/or item images). If a difference between the estimated receptacle fill level derived from the receptacle bar feature and the estimated receptacle fill level derived from the additional data is lower than a set fill level accuracy threshold, the receptacle fill level derived from the receptacle bar feature is considered to be accurate. If a difference between the estimated receptacle fill level derived from the receptacle bar feature and the estimated receptacle fill level derived from the additional data is greater than a set fill level accuracy threshold, an alert can be generated to verify the respective receptacle to determine whether the receptacle was tempered with or whether the receptacle bar was blocked at a particular position. In some implementations, the current fill level of the receptacle, facility events that can affect the filling speed of a receptacle, and historical receptacle data can be used as an input for a machine learning algorithm to estimate a future expected fill level of the receptacle within a set period of time (e.g., next 6 hours, 12 hours, 24 hours, 2 days, 3 days, and/or 7 days). The historical receptacle data includes a frequency of receptacle use, a disposed item type, one or more facility events, and an occupancy level of an associated facility. The receptacle status of each receptacle within the facility can be displayed (as described with reference to FIGS. 2 and 3) on a graphical user interface of a depositing system (e.g., depositing system 102A, 102B described with reference to FIGS. 1A and 1B) and/or a user device (e.g., user device 104 described with reference to FIG. 1A). The current receptacle status can include an availability of the receptacle to store additional items based on a current fill level of the receptacle and, optionally, based on an estimated fill level within a future period of time. In some implementations, determining the availability of the receptacle to store additional items includes comparing the current fill level of the receptacle to a fill level threshold. For example, if the current fill level of the receptacle is below a set threshold (e.g., 90% of total fill level of the receptacle), the receptacle is identified as being available for depositing additional items. If the current fill level of the receptacle is above the first threshold (e.g., 90% of total fill level of the receptacle) but not completely filled, the receptacle is identified as being critically full and can be available for particular deposits of particular items (e.g., highly regulated items identified as necessary to be deposited within a limited period of time). If the current fill level of the receptacle reached a maximum filling level (e.g., 100% of total fill level of the receptacle) the receptacle is identified as unavailable for any type of item deposits. In some implementations, the identification of the receptacle can be based on a physical characteristic (e.g., height, width, volume, and/or weight) of the item (e.g., receptacle may not be full but it may not have sufficient available volume to accept the item to be returned.

At 406, a request to deposit one or more items is received, by the computing system. The request can be generated by a user input entered by interacting a depositing system (e.g., depositing system 102A, 102B described with reference to FIGS. 1A and 1B) and/or a user device (e.g., user device 104 described with reference to FIG. 1A).

At 408, a receptacle available to deposit the items is identified, by the computing system, for depositing the items based on availability levels of receptacles, a distance to receptacles, and/or item types. In some implementations, determining the availability of receptacles to deposit items includes processing receptacle data, such as comparing current fill level of receptacles to a fill level threshold. For example, if the current fill level of the receptacle is below a set threshold (e.g., 90% of total fill level of the receptacle), the receptacle is identified as being available for depositing additional items. Identifying a receptacle to deposit the items can include identifying available receptacles within a particular distance from the source of user input. For example, if the user input was entered on a depositing system, the receptacle(s) within the depositing system are first analyzed to determine availability to deposit the items relative to the fill level of the receptacle(s). If no receptacle within the depositing system is available for depositing the items, additional receptacles are identified and ranked based on a distance between the depositing system receiving the user input and the location of the available receptacles. If the user input was entered on a user device and multiple depositing systems are identified as including available receptacles for depositing the items, the depositing system corresponding to the receptacle with the lowest filling level is identified to be used for depositing the items. Identifying a receptacle to deposit the items can include matching a receptacle type to an item type. For example, if the item to be deposited is a highly regulated item, receptacles within the shortest distance are identified. For deposition of highly regulated items, critically high fill level are also considered to identify the nearest available receptacle to deposit the highly regulated item.

At 410, a trigger to activate item deposition at the identified receptacle is generated, by the computing system. Item deposition can be activated by displaying a prompt to deposit the item(s). For example, a user interface (e.g., user interface 108 of the depositing system 102A, 102B or the display 122 of user device 104 described with reference to FIG. 1A) can generate a visual and/or audio command for the user to deposit the item(s). The prompt can include an identifier of an available receptacle (and an associated map to guide the user to the available receptacle, if the receptacle is away from the location where the user input was entered) on a graphical user interface. Item deposition can be activated by opening an entry mechanism (e.g., the entry mechanism 114c, described with reference to FIGS. 1A and 1B), to enable the user to deposit the items in the receptacle. The entry mechanism can enable deposition of items in the receptacle in a manner that triggers a movement of a variable length bar that is monitored to derive the change in the fill level of the receptacle.

At 412, items deposited in the receptacle are being detected, by the computing system. In some implementations, in response to receiving the signal indicative of the items being deposited in the receptacle, a controller of the depositing system can activate a settling feature of the receptacle to optimize an arrangement of the items within the receptacle, by minimizing a volume occupied by the items within the receptacle. The opening of the entry mechanism or a completion of a settling process can trigger activation of the receptacle sensors to detect a change in receptacle fill level in response to the deposition of the items. The sensors (e.g., sensors 134a-e described with reference to FIGS. 1A and 1B) attached to a receptacle can detect a change in fill level (e.g., change in bar length, angle, and or item weight) or an updated fill level after the deposition of the items (e.g., current bar length, current bar angle, and or current item weight). The receptacle bar feature data (bar length and/or an angle between the bar and a vertical axis intersecting a pivoting element used to attach the bar to a portion of the receptacle) can be automatically processed to determine updated receptacle data including an updated fill level. The updated fill level derived from the receptacle bar feature data can be compared to the updated fill level derived from the additional data (e.g., images, item identifiers associated with item volume and weight, and/or item weight of the items deposited within a respective receptacle) to determine an accuracy of the receptacle data. The receptacle data identified as accurate can be automatically transmitted to a central data processing system of the facility (e.g., data processing system 110, described with reference to FIG. 1A). In some implementations, a controller of a depositing system can transmit a signal for the entry mechanism to be closed after the items were deposited to prevent unmonitored handling of items deposited in the receptacle.

At 414, a status of a receptacle is updated, by the computing system, based on the receptacle updated fill level. Updating the status of the receptacle includes a comparison of the updated fill level to one or more set thresholds. For example, a comparison to a first threshold enables a differentiation between available receptacles and partly filled receptacles, where the fill level is below the first threshold. A receptacle with an updated fill level that is greater than the first threshold but below a second threshold can be identified as a critically filled receptacle. A receptacle with an updated fill level that is approximately equal to the second threshold can be identified as being a completely filled (unavailable) receptacle.

At 416, a status of the receptacle can be transmitted, by the computing system, to a central data processing system of the facility (e.g., data processing system 110, described with reference to FIG. 1A). In some implementations, status transmission includes generation of an alert. The alert can indicate that a receptacle is completely filled (unavailable) receptacle or critically filled or a particular item type (e.g., highly regulated substance) was deposited in a particular receptacle (identified using a receptacle and/or location identifier). The alert can include a visual and/or audio alert. In some implementations, generating the alert includes adding a textual note and/or a highlighting feature associated with the receptacle identified as having a critically high filling level or a completely filled level. In some implementations, the alert is displayed on a user interface (e.g., user interface 108 of the depositing system 102A, 102B or the display 122 of user device 104 described with reference to FIG. 1A). The receptacle status can be transmitted to an indicator of a respective depositing system (e.g., indicator 146, described with reference to FIG. 1A), to illustrate a fill status of the receptacle. In some implementations, the indicator indicates the fill status of the receptacle using a color code that can be visible for a user of the depositing system. In some implementations, the controller transmits an alert based on the updated fill status of the receptacle, such as to a display of the depositing system (e.g., the input user interface 108). The alert may include a visual, audio, audiovisual, tactile, and/or the like, indicator that indicates the fill status of the receptacle.

At 418, future receptacle fill levels is predicted, by the computing system or the data processing system, based on the current fill level of the receptacles (being updated each time additional items are deposited in receptacles), based on historical receptacle fill levels (including filling patterns), and based on facility events associated with triggering a modification of a filling speed of receptacles. The future receptacle fill levels can be predicted using a machine learning model that is continuously updated to increase the accuracy of the estimates of future receptacle fill levels for each receptacle within the facility. In some implementations, the threshold values can be updated based on the machine learning model, such that a critical filling level matches an emptying schedule and extends (optimally to a continuous, uninterrupted time) the availability of receptacles to receive additional items. The future receptacle fill levels can be generated to predict fill levels corresponding to a particular period of time expressed in hours or days. In some implementations, the future receptacle fill levels can be displayed on a user interface (e.g., user interface 108 of the depositing system 102A, 102B or the display 122 of user device 104 described with reference to FIG. 1A).

At 420, a receptacle emptying plan can be generated, by the computing system or the data processing system, for all receptacles within the facility. The receptacle emptying plan can be generated based on the current receptacle fill level and/or future predicted receptacle fill levels relative to an availability of receptacle controllers. The receptacle emptying plan can be automatically updated each time an additional item is deposited in receptacles or any parameter that can affect the emptying plan is modified. The receptacle emptying plan can be adjusted in anticipation of planned facility events expected to increase the filling speed of the receptacles faster than in the absence of facility events. The receptacle emptying plan can be adjusted to prioritize emptying receptacles where highly regulated items were deposited. The receptacle emptying plan can be adjusted to reset an emptying time of a receptacle after a receptacle was emptied. The receptacle emptying plan can include verification of receptacles where a nonconformity (mismatching receptacle filling data detected by a plurality of sensors or a mismatch between detected receptacle filling data and predicted receptacle filling data) was identified.

At 422, the receptacle emptying plan can be transmitted to a user device to be displayed on a user interface (e.g., user interface 108 of the depositing system 102A, 102B or the display 122 of user device 104 described with reference to FIG. 1A) to enable receptacle controllers to implement the generated plan. In some implementations, the display of the receptacle emptying plan includes a display of all the receptacles within a (portion of a) facility relative to a map of the facility, as described with reference to FIGS. 2 and 3. In some implementations, the display of the receptacle emptying plan enables a user input to be entered to modify the receptacle emptying plan, based on one or more prioritizing parameters. In some implementations, the display of receptacle emptying plan can include generating a reminder and/or an alert to empty at least one receptacle within the facility.

The system for synchronization of emptying schedule of receptacles with respective receptacle fill levels can help to prevent delays with item deposition, preventing such delays to interfere with a schedule of the receptacle user (health care provider). The detection of a receptacle fill level including the sensor assembly described herein may accurately determine the receptacle fill level based on length detection of a variable length bar, which helps to improve control of deposition of items within receptacles and a receptacle emptying plan to prevent allowing receptacles to be filled until they cannot be used for deposition of additional items.

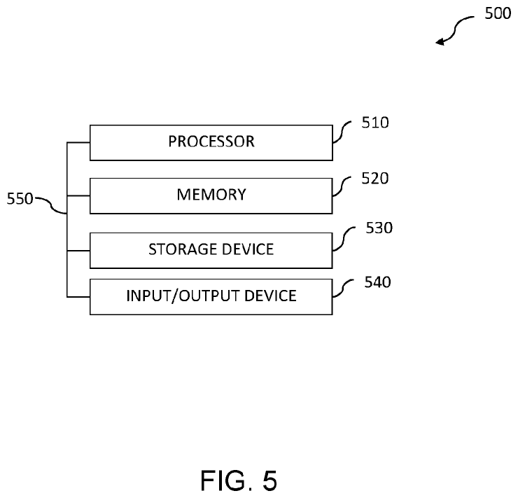

FIG. 5 depicts a block diagram illustrating a computing system 500 consistent with implementations of the current subject matter. Referring to FIGS. 1 and 5, the computing system 500 can be specifically configured for determining a fill level of a receptacle of a depositing system with a computing system, a display, and/or any components therein.

As shown in FIG. 5, the computing system 500 can include a processor 510, a memory 520, a storage device 530, and input/output devices 540. The processor 510, the memory 520, the storage device 530, and the input/output devices 540 can be interconnected via a system bus 550. The processor 510 is capable of processing instructions for execution within the computing system 500. Such executed instructions can be implemented by one or more components of, for example, the depositing system 102A, 102B. In some example implementations, the processor 510 can be a single-threaded processor. Alternatively, the processor 510 can be a multi-threaded processor. The processor 510 is capable of processing instructions stored in the memory 520 and/or on the storage device 530 to present graphical information for a user interface provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a hard disk device, an optical disk device, or a tape device, or other suitable persistent storage means. The input/output device 540 provides input/output operations for the computing system 500. In some example implementations, the input/output device 540 includes a keyboard and/or pointing device. In various implementations, the input/output device 540 includes a display unit for displaying graphical user interfaces.

According to some example implementations, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some example implementations, the computing system 500 can be used to execute various interactive computer software applications that can be used for organization, analysis and/or storage of data in various formats. Alternatively, the computing system 500 can be specifically configured to execute software applications. These applications can be used to perform various receptacle fullness monitoring functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.).

One or more aspects or features of the subject matter described herein can be realized in specifically configured digital electronic circuitry, integrated circuitry, application-specific integrated circuit (ASIC), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

As used herein a “user interface” (also referred to as an interactive user interface, a graphical user interface or a user interface or UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, NET™, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the receptacle via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, or the like. A message may, in some implementations, include a signal utilized to transmit one or more representations of the information. While recited in the singular, it will be understood that a message may be composed, transmitted, stored, received, etc. in multiple parts.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In some implementations, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

1. A computer-implemented method, comprising:
receiving, by one or more processors, receptacle data associated with filling levels of one or more receptacles of depositing systems located at a facility;
receiving, by the one or more processors, a request to deposit an item;
identifying, by the one or more processors and based at least on the receptacle data, a first receptacle of the one or more receptacles for receiving the item; and
generating, by the one or more processors, a user interface displaying the first receptacle as available to receive the item.

2. The computer-implemented method of claim 1, further comprising:
detecting, by the one or more processors, depositing of the item at the first receptacle.

3. The computer-implemented method of claim 2, further comprising:
receiving, by the one or more processors, an updated filling level of the first receptacle after detection of disposal of the item at the first receptacle.

4. The computer-implemented method of claim 1, further comprising:
predicting, by the one or more processors, a future filling level of the one or more receptacles of the depositing systems in the facility by applying a machine learning model trained to process the receptacle data associated with filling levels and historical receptacle data; and
using the future filling level to update threshold values by applying the machine learning model trained to process the receptacle data associated with filling levels and historical receptacle data.

5. The computer-implemented method of claim 4, wherein the historical receptacle data comprises a frequency of receptacle use, a disposed item type, one or more facility events, and an occupancy level of an associated facility.

6. The computer-implemented method of claim 4, further comprising:
generating, by the one or more processors, a receptacle emptying plan based on the future filling level of the one or more receptacles of the depositing systems in the facility.

7. The computer-implemented method of claim 6, further comprising:
providing, by the one or more processors, for display the receptacle emptying plan.

8. The computer-implemented method of claim 6, wherein the receptacle emptying plan comprises a schedule to empty the one or more receptacles of the depositing systems in the facility within a time period.

9. The computer-implemented method of claim 8, wherein the receptacle data comprises a receptacle bar feature that is variable with a filling level of a respective receptacle, the receptacle bar feature comprising at least one of a length, a position, and an angle of the receptacle bar.

10. The computer-implemented method of claim 9, wherein the request to deposit the item is generated by a second depositing system comprising a second receptacle and identifying the first receptacle of the one or more receptacles for depositing the item comprises:
determining, by the one or more processors, a distance between the first receptacle and the second receptacle, the second receptacle having a second filling level above a threshold and the first receptacle having a first filling level below the threshold.

11. The computer-implemented method of claim 10, further comprising:
generating, by the one or more processors, an alert indicating that the second receptacle has the second filling level above the threshold.

12. The computer-implemented method of claim 11, further comprising:
generating, by the one or more processors, an alert indicating one or more nonconformities of filling levels of one or more receptacles.

13. The computer-implemented method of claim 12, wherein the one or more nonconformities are associated to mismatching receptacle filling data detected by a plurality of sensors or a mismatch between detected receptacle filling data and predicted receptacle filling data.

14. The computer-implemented method of claim 12, wherein the receptacle data associated with the filling levels of the one or more receptacles is detected by one or more sensors comprising at least one of a pressure sensor, a camera, a magnetic sensor, a radio frequency sensor, and a light sensor.

15. The computer-implemented method of claim 14, wherein the item comprises an identifier code, and wherein the computer-implemented method further comprises receiving, by the one or more processors, the identifier code from a scanning device associated with the first receptacle.

16. The computer-implemented method of claim 15, further comprising: enabling, by the one or more processors, access to the first receptacle by activating an opening of the first receptacle for depositing the item.

17. The computer-implemented method of claim 16, wherein the depositing systems comprise a user interface, wherein the user interface provides information and/or instructions related to the second receptacle selected for disposing the item.

18. The computer-implemented method of claim 17, wherein the depositing systems comprise a medication depositing system.

19. The computer-implemented method of claim 18, wherein the instruction to proceed with depositing the item at the first receptacle comprises an indication of a location of the first receptacle.

20. The computer-implemented method of claim 19, wherein identifying is based on a physical characteristic of the item relative to an available volume of the one or more receptacles associated with the filling levels of the one or more receptacles.

21. (canceled)

22. (canceled)

23. (canceled)
